(19) United States
(12) Patent Application Publication
(10) Pub. No.: US 2009/0198048 A1
(43) Pub. Date: Aug. 6, 2009

(54) GLYCEROSE SYNTHESIS

(75) Inventors: Thomas Stephen Winowiski, Mosinee, WI (US); Jerry Daniel Gargulak, Ringle, WI (US)

Correspondence Address: IPLM GROUP, P.A., POST OFFICE BOX 18455, MINNEAPOLIS, MN, 55418, US

(73) Assignee: LignoTech USA, Inc.

(21) Appl. No.: 12/234,462

(22) Filed: Sep. 19, 2008

Related U.S. Application Data
(60) Provisional application No. 61/026,335, filed on Feb. 5, 2008.

(51) Int. Cl. C07H 1/00

(52) U.S. Cl. 536/124

(57) ABSTRACT

Glycerose is prepared by partially oxidizing biodiesel-derived glycerol to provide a glycerose liquor containing glycerose and other side products of the glycerol oxidation reaction. The glycerose liquor may be combined without further isolation, purification or drying with a protein-containing fodder or fodder precursor to provide a rumen undegradable protein-containing ruminant feed product.

### CROSS-REFERENCE TO RELATED APPLICATIONS

This application claims priority from U.S. provisional patent application Ser. No. 61/026,335 filed Feb. 5, 2008, the disclosure of which is incorporated herein by reference.

### FIELD

This invention relates to glycerose (viz., glyceraldehyde) and its manufacture.

### BACKGROUND

Glycerol has recently become readily available as a byproduct from biodiesel production. At present, approximately one kg of glycerol is obtained for every 9 kg of biodiesel. This has led to a global glycerol oversupply, with some of the excess glycerol being disposed of via incineration. Recent initiatives to decrease reliance on fossil fuels (for example, EU directive 2003/30/EC which targets 5.75% incorporation of biofuels by 2010) may aggravate the glycerol oversupply problem.

### SUMMARY OF THE INVENTION

Biodiesel-derived glycerol provides an inexpensive and very useful starting material for making glycerose, and especially for making concentrated glycerose solutions where a finished product with high purity is not required. The glycerose forms as a partial oxidation product of glycerol, in a glycerose liquor containing the glycerose and other side products of the glycerol oxidation reaction. The glycerose may be combined without further isolation, purification or drying with a protein-containing fodder or fodder precursor to provide ruminant feeds and feed supplements in which the feed protein and glycerose react via non-enzymatic browning. The resulting feed product contains rumen undegradable protein (RUP) which may also be referred to as bypass protected protein or rumen-protected protein. RUP resists degradation while in the ruminal tract but remains digestible when in the post-rumen tract.

Glycerose appears to be highly reactive towards protein-containing fodder. Other species in the glycerose liquor may be reactive towards protein-containing fodder as well. The reaction may take place more rapidly, or at lower addition levels, or at lower temperatures than is the case when reacting protein-containing fodder with other bypass protection reagents such as spent sulfite liquors.

The present invention provides in one aspect a method for making glycerose, which method comprises the steps of:
a) obtaining glycerol from a biodiesel manufacturing process; and
b) partially oxidizing the glycerol to provide a glycerose liquor containing glycerose and other side products of the oxidation reaction.

The invention provides in another aspect a method for making glycerose, which method comprises the step of combining a glycerol solution with an oxidizing agent and a ferrous ion catalyst to provide a reaction mixture containing more than 10 wt. % glycerol and less than 1000 ppm catalyst. This provides a concentrated glycerose solution containing reduced catalyst residue and having particular suitability for use in making RUP-containing ruminant feeds. The resulting feed product forms rapidly, requires relatively little drying time and contains limited metallic residue.

The invention provides in yet another aspect a method for making glycerose, which method comprises the step of combining a glycerol solution with an oxidizing agent, a catalyst and a chelating agent. The chelating agent discourages excessive scum formation which might occur if ferrous ions form salts with fatty acids that may be present in impure glycerol.

By replacing current bypass protection reagents for making RUP-containing ruminant feeds with the disclosed partial oxidation product, valuable use can be made of a surplus biodiesel waste byproduct which might otherwise be incinerated or used for low-value alternative uses.

### BRIEF DESCRIPTION OF THE DRAWING

FIG. 1 is a schematic view of one embodiment of the disclosed manufacturing process;

FIG. 2 is a graph showing glycerose conversion at various peroxide:glycerol ratios; and

FIG. 3 is a graph showing glycerose concentration at various peroxide:glycerol ratios.

### DETAILED DESCRIPTION

Unless the context indicates otherwise the following terms shall have the following meaning and shall be applicable to the singular and plural:

The terms “a,” “an,” “the,” “at least one,” and “one or more” are used interchangeably. Thus a reaction mixture that contains “a” protein-containing fodder may include “one or more” protein-containing fodders.

The term “crude glycerin” means an impure byproduct of biodiesel manufacturing, containing for example about 45 to about 85 wt. % glycerol.

The term “fodder” means a material suitable for feeding ruminant animals.

The term “fodder precursor” means a material (for example, crushed seeds, extracts, syrups, leaves, grasses, stalks or roots) that is subjected to one or more further processing steps or combined with one or more other materials to form a fodder product.

The term “glycerose liquor” means a liquid mixture containing glycerose and other side products of a glycerol oxidation reaction.

The term “impure”, when used with respect to a sample containing a desired chemical, means that the sample contains less than 85 wt. % of the desired chemical.

The term “partial oxidation product”, when used with respect to a material made from glycerol, means a partially but incompletely oxidized reaction product of glycerol and an oxidizing agent.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “ruminally inert”, when used with respect to a protein or lipid, means that the interaction of the protein or lipid with rumen bacteria is reduced or prevented, and the protein or lipid is rendered available for digestion and absorption in the post-rumen gastrointestinal tract of a ruminant.

The recitation of a numerical range using endpoints includes all numbers subsumed within that range (e.g., 1 to 5 includes 1, 1.5, 2, 2.75, 3, 3.80, 4, 5, etc.). The recitation of sets of upper and lower endpoints (e.g., at least 1, at least 2, at least 3, and less than 10, less than 5 and less than 4) includes all ranges that may be formed from such endpoints (e.g., 1 to 10, 1 to 5, 2 to 10, 2 to 5, etc.).

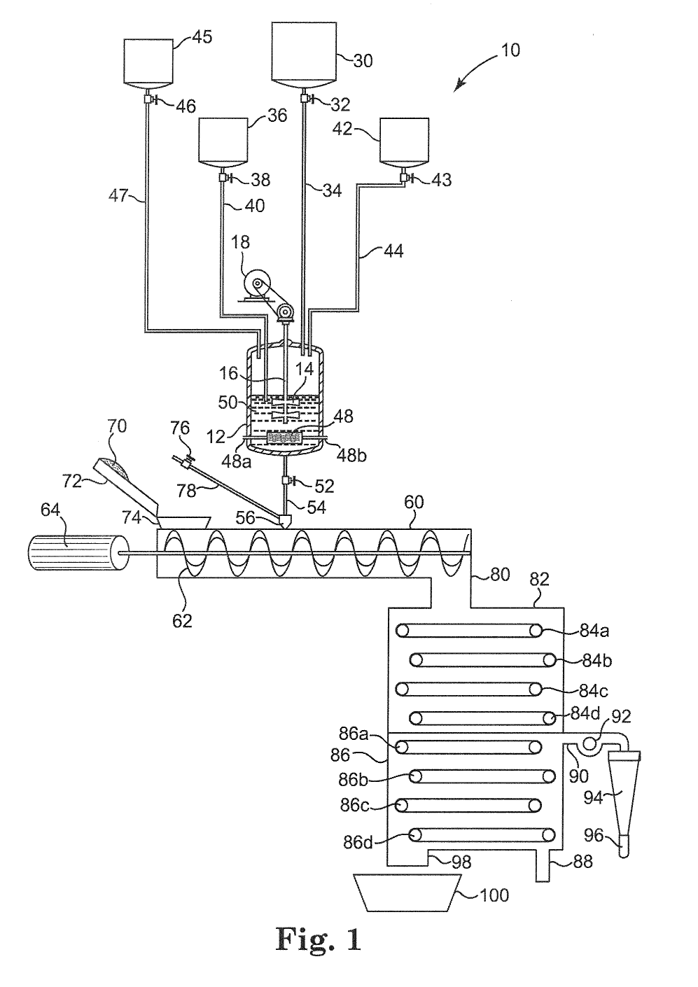

FIG. 1 shows an exemplary schematic view of one embodiment of the disclosed manufacturing process. Apparatus 10 includes an oxidation reactor 12 for making glycerose in the form of a glycerose liquor. Reactor 12 is equipped with an impeller 14 mounted on shaft 16 and driven by a motor 18. Glycerol (desirably obtained in concentrated solution form as a byproduct of biodiesel production) stored in vessel 30 is regulated by valve 32 and fed to reactor 12 through conduit 34. An oxidizer such as hydrogen peroxide stored in vessel 36 may be regulated by valve 38 and fed to reactor 12 through conduit 40. A catalyst solution such as a solution of ferrous sulfate stored in vessel 42 may be regulated by valve 43 and fed to reactor 12 through conduit 44. A chelating agent solution such as a solution of ethylene diamine tetraacetic acid (EDTA) stored in vessel 45 may be regulated by valve 46 and fed to reactor 12 through conduit 47. The reaction is generally exothermic, and a heat exchanger 48 supplied with inlet and outlet lines 48a and 48b connected to a suitable coolant source (not shown in FIG. 1) may be used to control the temperature in reactor 12. At the completion of the oxidation reaction, glycerose liquor 50 may be removed from reactor 12 by opening valve 52 and stored or shipped as needed.

An especially desirable use for the resulting glycerose-containing glycerose liquor is to combine the liquor with fodder or a fodder precursor to make RUP-containing ruminant feeds. Further details regarding such feeds may be found in copending U.S. Patent Application No. (Attorney Docket No. 250-P-223USU1) filed even date herewith, the disclosure of which is incorporated herein by reference. Liquor 50 may be reacted directly with fodder or a fodder precursor by feeding liquor 50 through conduit 54 and steam atomizing mixing valve 56 and thence into mixing chamber 60. Chamber 60 includes a rotating auger 62 powered by motor 64. Protein-containing fodder or fodder precursor such as soybean meal 70 enters chamber 60 via chute 72 and inlet 74. A heating source such as steam regulated by valve 76 enters mixing valve 56 via conduit 78 and thereby heats the fodder or fodder precursor. The heated, auger-mixed combination of fodder or fodder precursor 70 and glycerose liquor 50 exits chamber 60 via outlet 80 and enters heated conditioning chamber 82 where the mixture passes along a series of conveyor belts such as belts 84a, 84b, 84c, 84d, etc. for a period of time sufficient to carry out to a desired extent a non-enzymatic browning reaction between the glycerose liquor and the fodder or fodder precursor. The browned product may be cooled in cooling chamber 86 by passing it along a series of conveyor belts such as belts 86a, 86b, 86c, 86d, etc. Cooling may be assisted using a stream of ambient or chilled air which enters chamber 86 at air inlet 88 and exits at air outlet 90, passing through fan 92, cyclone 94 and filter bag 96. The cooled rumen undegradable protein-containing feed product exits chamber 86 via feed outlet 98 and may be collected in hopper 100 or another suitable vessel or shipping container.

The disclosed partial oxidation products may be in pure or impure form, may represent a mixture of species, and may include dextrorotatory, levorotatory or mixed enantiomers, epimers, dimers or other rearrangement products. The disclosed glycerose liquor appears to include one or more aldehyde-functional species such as D-, L- or D,L-glycerose, and may in addition contain one or more keto-functional species such as dihydroxyacetone, or one or more carboxy-functional species such as glyceric acid or hydroxypyruvic acid. An enediol intermediate formed by rearrangement of glycerose to dihydroxyacetone (or vice-versa) may also be present. The oxidation products may include hydroxyl groups (e.g., 2, 3 or 4 such groups), ether linkages or ring structures. Species which may be present include those shown for example in Scheme 1 at page 21 of Yaylayan et al., “Investigation of DL-glyceraldehyde-dihydroxyacetone interconversion by FTIR spectroscopy”, Carbohydrate Research 318 (1999) 20-25, in Scheme 1 at page 400 of Porta et al., “Selective oxidation of glycerol to sodium glycerate with gold-on-carbon catalyst: an insight into reaction selectivity”, Journal of Catalysts 234 (2004 397-403), and in Scheme 2 at page 4436 of Pagliaro et al. “From Glycerol to Value-Added Products”, Angew. Chem. Int. Ed., 2007, 46, 4434-4440 and at page 166 of Garcia et al., “Chemoselective catalytic oxidation of glycerol with air on platinum metals”, Applied Catalysis A: General 127 (1995) 165-176.

The partial oxidation products desirably are obtained by oxidizing crude glycerin or other impure but relatively concentrated glycerol source. A variety of oxidation methods may be employed, including reacting glycerol with an oxidizing agent in the presence of a suitable catalyst; reacting glycerol with oxygen or air using water as a solvent; or reacting glycerol via enzyme oxidation, bacterial fermentation, catalytic oxidation or combinations thereof. Representative oxidation methods may be adapted, for example, from Witzemann, JACS 36, p. 2223-33 (1914) where glycerol is reacted with hydrogen peroxide in the presence of ferrous sulphate to make dl-glyceric aldehyde; from Garcia et al., supra, and Porta et al., supra; from U.S. Pat. No. 4,353,987 where glyceraldehyde is produced from a methanol dehydrogenase enzyme; and from U.S. Pat. No. 5,998,608 where sodium arbinoate is converted to D-glyceraldehyde using cobalt chloride hexahydrate and hydrogen peroxide. The disclosed partial oxidation reaction desirably is controlled so that glycerose is produced at high yield without further reaction to form more completely oxidized species such as glyceric acid.

When impure glycerol starting solutions are employed, their composition may vary depending upon the glycerol supply source and other factors such as the presence of side products. For example, some biodiesel-derived glycerol starting solutions appear to be more susceptible than others to foaming when oxidized, perhaps due the presence of larger amounts of free fatty acids. An increased likelihood of foaming may also be observed when using highly concentrated glycerol starting solutions. However, the starting solution desirably has a relatively high initial glycerol concentration, e.g., more than more than 10 wt. %, more than 30 wt. %, more than 50 wt. % or more than 60 wt. % glycerol. Highly concentrated glycerol starting solutions can provide partially oxidized glycerose liquors containing glycerose in high concentrations. These glycerose-rich liquors may in turn permit the manufacture of RUP-containing ruminant feeds requiring less heating. Biodiesel-derived crude glycerin (whose glycerol concentration may be as much as 70 to 80 wt. % or more) is an especially preferred starting solution. For some glycerol starting solutions it may be useful to alter or adjust the chosen oxidation process to reduce or compensate for undesired side reactions, exotherm, foaming or other artifacts which may be observed using that particular starting solution.

A preferred method for making the disclosed partial oxidation products involves reacting a concentrated glycerol starting solution with an oxidizing agent such as hydrogen peroxide in the presence of a suitable catalyst (for example, Fenton's reagent) and a suitable chelating agent (for example, EDTA). Other chelating agents such as citric acid could also be used, but may also have an increased tendency to interact with ferrous ion and fatty acids in some crude glycerins, e.g., by causing scum formation. The catalyst, chelating agent or both may be predissolved in water or other suitable solvents or added directly to the glycerol solution. Use of direct addition will reduce the extent of glycerol solution dilution and may help increase the extent or rate of glycerose formation. The oxidation reaction may be performed at any convenient temperature or pressure, e.g., at ambient or elevated temperature and ambient, reduced or elevated pressure. Preferably the oxidation reaction is performed at a temperature less than about 90° C., less than about 70° C. or less than about 50° C. Sufficient oxidant desirably is employed so as to attain the desired degree of reaction, for example about 0.2 to about 2 moles oxidant, about 0.3 to about 1.5 moles oxidant, about 0.5 to about 1.2 moles oxidant or about 0.8 to about 1 moles oxidant per mole of glycerol. Production costs may increase as more oxidant is used, but increasing the oxidant:glycerol ratio may also improve the extent or rate of glycerose formation.

Sufficient catalyst to achieve the desired rate of reaction desirably is also employed. For example, when using an iron-containing catalyst (e.g., a ferrous ion catalyst), the catalyst amount may be e.g., about 500 to about 3000 ppm iron, about 500 to about 2000 ppm iron or about 500 to about 1500 ppm iron based on the total reaction mixture weight. When a chelating agent is employed, the chelant may for example be used in an amount sufficient to complex all of the catalyst, e.g., at up to about twice the catalyst weight. The reaction desirably is conducted under acidic condition, e.g., at a pH less than 7, less than 6, less than 5, less than 4 or less than 3. The rate or extent of the oxidation reaction may be monitored in a variety of ways including monitoring elapsed reaction time or reaction mixture temperature, or by performing spectroscopic or titrimetric measurements on samples withdrawn from the reaction mixture to monitor the appearance of reaction products or reactive groups or the disappearance of reactants or reactive groups. The reaction desirably is halted before the glycerose is converted to other products such as glyceric acid, dihydroxyacetone and the like. The reaction thus should not be taken as far as is the case in some of the above-mentioned glycerol oxidation procedures, e.g., as in Porta et al.

A variety of fodders may be combined with the glycerose-containing glycerose liquor to make the disclosed RUP-containing ruminant feeds. Representative fodders include soybeans, lentils, cowpeas, peas, kidney beans, lima beans and other beans; clovers; seeds and seed products including canola, cottonseed, flaxseed, linseed, mustard seed, peanut, safflower, sesame and sunflower; cereals and other crops including alfalfa, barley, maize (corn), millet, oats, sorghum and wheat; by-product protein feedstuffs such as distillers and brewers grains, feather meal, fish products, milk products, poultry products and sugar beet waste; seaweed; and mixtures thereof. The fodder may already be in a ruminant-consumable form or may be a fodder precursor intended for further processing into a fodder product. For example, the fodder or fodder precursor may be in the form of whole or crushed seeds or grains, meal, flour, oil cake, press cake, pellets and syrup. The fodder may be a commercially available feed such as a high protein feed (for example, RALLY™ high energy lipid ration or METAPRO™ lactation rations, both from Lake O'Lakes Purina Feed LLC).

The reaction between the glycerose liquor and fodder or fodder precursor may be performed using any convenient mixing technique, mixing ratio, temperature and time sufficient to provide increased resistance to rumen degradation for protein, lipids or both protein and lipids in the fodder or fodder precursor and thereby make them ruminally inert. A feed product containing ruminally inert protein may for example be prepared by combining the glycerose liquor and fodder or fodder precursor by spraying or dripping the glycerose liquor onto the fodder or fodder precursor, or by using a mixing auger or other suitable device to combine the ingredients. The mixing ratio may conveniently be expressed in terms of the glycerose liquor add-on rate, calculated by determining the aldehyde content of the glycerose liquor (depending on the technique, doing so may also measure the amount of ketones such as dihydroxyacetone) and comparing the aldehyde amount expressed as the weight of an equivalent amount of pure glycerose to the fodder or fodder precursor dry matter weight. The reaction between the glycerose liquor and fodder or fodder precursor is believed generally to be a 1 mole to 1 mole reaction between free carbonyl groups in the glycerose liquor and free amino groups in the fodder or fodder precursor. For example, the pure glycerose equivalent amount may be at least about 0.25 wt. %, at least about 0.5 wt. %, at least about 0.75 wt. %, at least about 1 wt. %, at least about 1.5 wt. % or at least about 2 wt. % of the fodder or fodder precursor dry weight. The pure glycerose equivalent amount may for example also be less than about 6 wt. %, less than about 5 wt. %, less than about 4 wt. %, less than about 3 wt. %, less than about 2.5 wt. % or less than about 2 wt. % of the fodder or fodder precursor dry weight.

Glycerose liquor may also be combined with a cracked or crushed oilseed body to provide a feed product containing ruminally inert lipid(s). The resulting product may include a substantial amount of small particles having a lipid interior and a coating formed of reaction products of glycerose liquor with proteinaceous membranes from oilseeds. The coating may encapsulate the lipid in a protective matrix, thereby forming a compartment of protected protein containing the lipid such that the entire compartment and its lipid content escape degradation by rumen bacteria yet are digestible in the small intestine or abomassum of a ruminant. Some of the ruminally inert lipid may be transferred in polyunsaturated form to the ruminant milk, and the formation of trans-fatty acids in the rumen by bacterial hydrogenation may be reduced. The feed product may for example be prepared by first selecting the desired oilseed or mixture of seeds and breaking the seed cuticle by mechanical cracking, e.g. using a roller mill. Any other suitable method for breaking or cracking the seed cuticle may be employed, while taking care not to release oil unduly during the crushing process. The oilseeds may optionally be dried before or after cracking. Typically, this is accomplished by heating with hot air. Dry seeds may more readily absorb the glycerose liquor into the seed interior. However, drying may also increase production costs. The cracked seeds may be treated with glycerose liquor by applying the liquor in any suitable manner, for example by spraying, dripping, mixing, steeping or any other convenient method. The glycerose liquor desirably is allowed or caused to penetrate the seed interior, and optionally assisted via the use of steam, hot air, microwave energy, externally applied heat or any other convenient heating source. Desirably, sufficient glycerose liquor is distributed within the oilseed so that at least thirty percent of the lipid content is rendered ruminally inert. The resulting product may include ruminally inert lipid bodies of about 0.5 to about 10 micrometers average diameter, with the actual range of diameters typically depending on the chosen oilseed. For example, the lipid bodies in soybeans may have a size range between about 0.5 and about 2 micrometers. The ruminally inert bodies may for example include the lipid in its in situ natural form surrounded by a shell layer of the reaction product of a protein and the glycerose liquor, with the ratio of reaction product to lipid being for example between about 1 and 35 wt. %. The ratio of the reaction product to oleosin proteins in the shell layer may for example be about 0.5 to about 40 wt. %. The reaction product may for example be more dense than the lipid layer and relatively thin, e.g., with a thickness less than 10% of the lipid body diameter. The product may be ground if desired. The lipid bodies in such a ground product desirably are sufficiently small so as to remain intact and ruminally inert.

Whether the feed product involves ruminally inert protein, ruminally inert lipids or both, the reaction between the glycerose liquor and the fodder or fodder precursor may be referred to as a browning reaction. Under some mixing and handling conditions browning might not be required or observed. The reaction may be performed at any convenient temperature, e.g., ambient or elevated temperature, so long as the fodder or fodder precursor does not undergo excessive thermal degradation. The reaction temperature may for example be at least about 20° C., at least about 25° C. or at least about 30° C. as determined by measuring the mixture temperature. The reaction temperature may also for example be less than about 130° C., less than about 120° C. or less than about 100° C. The reaction may also be performed at any convenient pressure, e.g., ambient, elevated or reduced pressure. Steam, externally applied heat, or both steam and externally applied heat may be used. Steam may also be employed to regulate the moisture content of the fodder or fodder precursor, or of the completed feed product, and as an aid to penetration into an oilseed interior. The feed product moisture content after heating may for example be about 6% to about 40% by weight. In another useful embodiment, the glycerose liquor and the fodder or fodder precursor are combined in an extruder or pelletizer and the frictional heat of mixing is relied upon to carry out the reaction. Where externally-applied heat is employed, the glycerose liquor and fodder or fodder precursor may for example be heated for at least about 5 minutes, at least about 10 minutes, at least about 15 minutes, at least about 30 minutes, at least about 45 minutes or at least about 1 hour. The total heating time may also for example be less than about two hours, less than about one hour, less than about 45 minutes, less than about 30 minutes, less than about 15 minutes or less than about 10 minutes. Heating may be discontinued and the mixture allowed to cool for a period of time (e.g., 30 minutes or more, one hour or more, or two hours or more) while further reaction (conditioning) takes place.

The amount of ruminally inert protein or lipid in a feed product can be tailored to a variety of situations, including the chosen feed, ruminant, feeding schedule and desired milk characteristics. For example, the chosen glycerose liquor add-on amount, heating times and temperatures may be evaluated in a variety of ways, including the use of in vitro or in situ feed studies such as those described in the above-mentioned U.S. Pat. Nos. 4,957,748, 5,023,091, 5,064,665 and 5,789,001 and in Published U.S. Patent Application No. US 2008/0152755 A1. The evaluation may examine RUP, rumen-undigested oil or fat (RUF), or combinations of RUP and RUF. The procedure shown below was employed at the FARME Institute (Homer, N.Y.) to evaluate RUP levels:

### FARME Institute in situ Testing Protocol

Feed samples are tested for digestibility in situ using high producing lactating dairy cows in an early to mid stage of lactation. The cows are fed a standard dairy cattle ration balanced according to 2001 NRC recommendations. Sample sets are run in triplicate using approximately 5 g dry matter (DM) in each subset. Coarse feed samples such as whole cottonseed and soybeans are coarsely chopped prior to weighing. The as-received or if need be chopped samples are weighed into a 10 cm×20 cm nitrogen-free porous polyester bags whose pore size is 50±15 micrometers. The bags are heat-sealed after filling. The bags are soaked in water to moisten the feed, and then incubated in the rumen of a single cannulated cow. After incubation for a specified time period (e.g., 16 hours), the samples are withdrawn, frozen for a minimum of 12 hours, thawed and machine rinsed with cold water. The rinsed samples are then fully dried in a convection drying oven for a minimum of 12 hours at 49° C. and weighed. Standard disappearance calculations are employed and coefficients of variation are determined for each sample. Outliers are not used in the final average if the coefficients of variation are greater than 10%. Chemical analysis of fat and protein is conducted on both the original feed samples and the digestive residues at the Dairy One Cooperative forage laboratory (Ithaca, N.Y.). Disappearance of protein is calculated by determining the grams of protein remaining after digestion and subtracting that quantity from the grams of protein placed into the in situ bag. The percentage of protein that has disappeared is described as rumen degradable protein (RDP). The percentage that remains is rumen undegradable protein (RUP). Disappearance of other nutrients is determined in a like manner.

The disclosed ruminant feed product desirably exhibits at least about a 25% reduction and more preferably at least about a 50% reduction in RDP compared to a control feed that has not been reacted with an oxidation product of glycerol. Expressed another way, the ruminant feed product desirably contains at least about 50%, at least about 60% or at least about 70% RUP as a percent of crude protein (CP). The feed product may be stored, packaged or further modified as desired (e.g., by pelletizing, or by adding at least one nutrient, medication, vitamin, etc.) prior to being administered to ruminant animals. Appropriate storage and packaging techniques and feed product modifications will be familiar to persons having ordinary skill in the art.

Ruminants to which the disclosed feed product may be administered include cattle (both dairy and beef cattle), goats, sheep, alpacas, antelope, bison, camels, deer, giraffes, llamas, water buffalo, wildebeest and yaks. The feed product typically will be administered as a feed supplement, e.g., at about 0.5 to about 20, about 1 to about 10 or about 1 to about 5% of the total feed intake. Administration amounts may be determined empirically and may be based on factors including weight gain, milk production, milk content or meat analysis.

The invention is further described in the following Examples, in which all parts and percentages are by weight unless otherwise indicated.

### EXAMPLE 1

### Glycerose Liquor Preparation

A 1.5 part quantity of ferrous sulfate heptahydrate catalyst was placed in an open reaction vessel equipped with a mechanical stirrer and chilled using an ice water bath. The catalyst was dissolved in 15 parts deionized water and 200 parts of a biodiesel-derived glycerol waste stream containing 85% glycerol obtained from Cargill Inc. (Minnetonka, Minn.). A second reaction mixture was similarly prepared using a reaction vessel whose temperature was controlled using an ambient temperature water bath instead of ice water. The two reaction vessels were designated as Cool (C) and Warm (W). A 50 part quantity of 35% hydrogen peroxide was added dropwise to each reaction vessel over a one hour period. The reaction vessels were allowed to stand for a 30 minute rest period during which the pH was measured and adjusted to 3.0 using 5N sodium hydroxide as needed and 20 parts of the reaction mixture were collected.

A portion of the collected reaction mixture was analyzed to determine the reducing sugar content and reported as a weight percent equivalent of reducing moieties in the sample compared to a known glucose standard. The standard was prepared by dissolving 0.1439 g dry glucose in 500 mL deionized water. A 5 mL aliquot (viz., a portion containing 1.439 mg glucose) was combined with 5 mL of Copper Reagent (prepared by dissolving 53 g Na₂HPO₄.7H₂0 and 40 g NaKC₄H₄O₆.4H₂O in about 70 ml of water, followed by 100 mL portions of 1N sodium hydroxide, 8 g CuSO₄.5H₂O, 180 g Na₂SO₄, and 0.7134 g KIO₃, and after everything had dissolved diluting the solution to 1 L) and heated to 100° C. for 40 minutes. The mixture was cooled, combined with potassium iodide and a starch indicator, and titrated with sodium thiosulfate. A net addition of 14.6 mL titrant was required compared to a blank prepared without glucose. The standard accordingly had a “Sugar Factor” of 0.0989 mg glucose/mL titrant. A portion of the collected reaction mixture sample (e.g., 1.01 g) was diluted to 500 mL and a 5 mL aliquot was combined with Copper Reagent, heated and titrated with thiosulfate in a similar manner. The net titrant required was multiplied by the Sugar Factor to determine the sample portion reducing sugar content.

The hydrogen peroxide addition, rest, sampling and analysis procedures were repeated four more times (for a total of five hydrogen peroxide additions in all) by adding a hydrogen peroxide amount corresponding to 25% of the amount of glycerol-containing biodiesel waste stream remaining in the reaction vessel. The material balance and reducing sugar results for the Cool and Warm reaction vessels are shown below in Tables 1A and 1B.

TABLE 1A
Cool Reaction Vessel Material Balance and Reducing Sugar Content

| | Amount in Reaction Vessel, Parts | | | | | | | |
|---|---|---|---|---|---|---|---|---|
| | | | Biodiesel | | | | | Percent |
| | | | Waste | | | | | Reducing |
| | | | Stream | | | | | Sugars |
| | | | (85% | | | | Collected | (Glucose |
| Time | FeSO₄•7H₂O | Water | Glycerol) | H₂O₂ | NaOH | Total | Sample | Equiv.) |
| 7:00 | +1.5 | +15 | +200 | +50 | | 267 | | |
| 8:15 | 1.5 | 15 | 200 | 50 | +4.21 | 271 | | |
| 8:30 | 1.4 | 13.9 | 185.2 | 46.3 | 3.9 | 251 | −20 | 10.60 |
| 8:30 | | | | +46.3 | | | | |
| 9:30 | 1.4 | 13.9 | 185.2 | 92.6 | 3.9 | 297.0 | | |
| 9:45 | | | | | 0 | | | |
| 9:55 | 1.3 | 13.0 | 172.8 | 86.4 | 3.6 | 277.0 | −20 | 15.80 |
| 10:00 | | | | +43.3 | | | | |
| 10:00 | 1.3 | 13.0 | 172.8 | 129.7 | 3.6 | 320.3 | | |
| 11:15 | | | | | +2.63 | | | |
| 11:20 | 1.2 | 12.1 | 162.0 | 121.6 | 5.9 | 300.3 | −20 | 18.00 |
| 11:30 | | | | +40.7 | | | | |
| 11:30 | 1.2 | 12.1 | 162.0 | 162.3 | 5.9 | 343.5 | | |
| 12:45 | | | | | +5.36 | | | |
| 12:50 | 1.1 | 11.4 | 152.5 | 152.8 | 10.6 | 323.5 | −20 | 17.40 |
| 13:00 | | | | +38.2 | | | | |
| 13:00 | 1.1 | 11.4 | 152.5 | 191.0 | 10.6 | 366.7 | | |
| 14:15 | | | | | +8.58 | | | |
| 14:20 | 1.1 | 10.8 | 144.2 | 180.6 | 18.1 | 346.7 | −20 | 15.60 |

TABLE 1B
Warm Reaction Vessel Material Balance and Reducing Sugar Content

| | Amount in Reaction Vessel, Parts | | | | | | | |
|---|---|---|---|---|---|---|---|---|
| | | | Biodiesel | | | | | Percent |
| | | | Waste | | | | | Reducing |
| | | | Stream | | | | | Sugars |
| | | | (85% | | | | Collected | (Glucose |
| Time | FeSO₄•7H₂O | Water | Glycerol) | H₂O₂ | NaOH | Total | Sample | Equiv.) |
| 7:00 | +1.5 | +15 | +200 | +50 | | 267 | | |
| 8:15 | 1.5 | 15 | 200 | 50 | +1.34 | 268 | | |
| 8:30 | 1.4 | 13.9 | 185.2 | 46.3 | 1.2 | 248 | −20 | 11.40 |
| 8:30 | | | | +46.3 | | | | |
| 9:30 | 1.4 | 13.9 | 185.2 | 92.6 | 1.2 | 294.4 | | |
| 9:45 | | | | | +3.68 | | | |
| 9:55 | 1.3 | 13.0 | 172.8 | 86.4 | 4.6 | 274.4 | −20 | 16.20 |
| 10:00 | | | | +43.3 | | | | |
| 10:00 | 1.3 | 13.0 | 172.8 | 129.7 | 4.6 | 321.1 | | |
| 11:15 | | | | | +3.31 | | | |
| 11:20 | 1.2 | 12.1 | 162.0 | 121.6 | 7.4 | 301.1 | −20 | 18.40 |
| 11:30 | | | | +40.7 | | | | |
| 11:30 | 1.2 | 12.1 | 162.0 | 162.3 | 7.4 | 344.9 | | |
| 12:45 | | | | | +5 | | | |
| 12:50 | 1.1 | 11.4 | 152.5 | 152.8 | 11.7 | 324.9 | −20 | 17.50 |
| 13:00 | | | | +38.2 | | | | |
| 13:00 | 1.1 | 11.4 | 152.5 | 191.0 | 11.7 | 367.8 | | |
| 14:15 | | | | | +9.6 | | | |
| 14:20 | 1.1 | 10.8 | 144.2 | 180.6 | 20.1 | 347.8 | −20 | 16.20 |

The individual samples from the Cool reaction vessel were identified as 1C, 2C, 3C and so on depending on whether the sample was collected at the first, second, third, etc. sample collection period shown in Table 1A. In similar fashion, the individual samples from the Warm reaction vessel were identified as 1W, 2W, 3W and so on depending on whether the sample was collected at the first, second, third, etc. sample collection period shown in Table 1B.

During the reaction, the Cool reaction vessel temperature was about 20-30° C. and the Warm reaction vessel temperature was about 45-55° C. A strong exotherm occurred when making the initial hydrogen peroxide addition, with less strong exotherms being observed for subsequent peroxide additions. The results in Tables 1A and 1B show that the reaction was not greatly affected by temperature, but that the Warm reaction provided somewhat greater reducing sugar content than the Cool reaction after corresponding reaction times.

The results in Tables 1A and 1B also show that the reducing sugar content peaked at around the third sample (3C or 3W) collection period. The ferrous ion catalyst may by that point have become sufficiently diluted so that further reaction would not take place without additional catalyst. The extent or rate of reaction may be improved by maintaining the concentration of ferrous ion at about 1000 ppm or more.

Analysis of sample 3W showed that a 5 mL aliquot of a diluted 1.01 g sample portion contained the equivalent of 1.87 mg glucose. Factoring in dilution, the original 1.01 g sample portion must have contained 187 mg of glucose equivalent, or 1.04 mmoles. At the time sample 3W was collected, the Warm reaction mixture contained 321 g of reactants, corresponding to 333 mmoles of glucose equivalent. This may be presumed to be 0.33 moles glycerose, plus unreacted glycerol, side reaction products such as dihydroxyacetone, and potentially other species as well. Based on the material balance shown in Table 1B, this 321 g reactant mixture was derived from 173 g of the biodiesel waste stream and 130 g of hydrogen peroxide, corresponding to about 1.6 moles glycerol and 1.3 moles hydrogen peroxide. This indicates that about 21% of the starting glycerol amount formed the target aldehyde, and that the extent of reaction might be further improved.

### EXAMPLE 2

### Feed Preparation

Portions of the collected samples from Example 1 were mixed with soybean meal (SBM) and heated to prepare an RUP-containing ruminant feed product. For each treatment a 150 g SBM portion which had been screened through a U.S. No. 8 sieve was combined with sufficient collected sample to add 1% glycerol or glycerol-derived oxidation products to the finished blend. The collected sample add-on amount was calculated on a dry matter basis with an assumption that the collected sample liquor contained unreacted glycerol, glycerose, dihydroxyacetone and possibly other byproducts. The SBM and collected sample portion were mixed in a plastic bowl in the amounts shown below in Table 2 and blended with an electric hand mixer. The resulting mixtures contained 80% dry matter (DM). Each mixture was placed in a glass jar, heated in a microwave oven for one minute, and then heated in a 105° C. oven for 15 or 30 minutes. The contents of the jars were spread onto paper, and allowed to cool and dry. The dried samples were sent to the FARME Institute for a 16 hour in situ protein degradation evaluation. The results are shown below in Table 2:

TABLE 2
Feed Treatments and Protein Evaluations

| | | Dosage, | | Crude | | |
|---|---|---|---|---|---|---|
| Run | Collected | % | Heating | Protein (CP), | RUP, % | RUP, % |
| No. | Sample | DM | Time, min | % of DM | of DM | of CP |
| 2-1 | 0 | 0 | 30 | 50.8 | 28.9 | 43.4 |
| 2-2 | 1W | 0.5 | 30 | 50.7 | 36.1 | 55.1 |
| 2-3 | 1W | 1 | 30 | 51.6 | 34.6 | 54.6 |
| 2-4 | 1C | 1 | 30 | 51.1 | 33.4 | 51.8 |
| 2-5 | 2W | 0.5 | 30 | 52.1 | 34.0 | 53.0 |
| 2-6 | 2W | 1 | 30 | 51.0 | 41.7 | 67.4 |
| 2-7 | 2C | 1 | 30 | 51.6 | 42.5 | 62.2 |
| 2-8 | 3W | 0.5 | 30 | 50.9 | 43.3 | 67.3 |
| 2-9 | 3W | 1 | 30 | 51.6 | 47.7 | 73.9 |
| 2-10 | 3C | 1 | 30 | 51.0 | 49.1 | 75.7 |
| 2-11 | 2W | 1 | 15 | 51.3 | 44.7 | 67.2 |
| 2-12 | 3W | 1 | 15 | 51.2 | 45.8 | 71.0 |

The results in Table 2 show that significant RUP increases were obtained by combining relatively low amounts of glycerose liquor with SBM at a very low heating temperature and for very short heating times. For example, addition of only 0.5% of collected sample 3W raised the RUP amount from 43.4% of Crude Protein (CP) for the control feed (heated but no glycerose liquor addition) to 67.3% of CP. Addition of 1% of collected sample 3W raised the RUP amount to 73.9% of CP using a 30 minute heating time, and to 71% of CP using only a 15 minute heating time. Comparison of Sample No. 2-6 with Sample No. 2-11, and of Sample No. 2-9 with Sample No. 2-12, suggests that the liquor has essentially completely reacted after 15 minutes and that little or no improvement may take place with an additional 15 minutes of heating.

### EXAMPLE 3

### High Temperature Glycerol Oxidation and Feed Preparation

One part ferrous sulfate heptahydrate catalyst was placed in a reaction vessel equipped with a thermometer and mechanical stirrer and dissolved in 10 parts deionized water. 100 Parts of a biodiesel-derived glycerol waste stream containing 80% glycerol obtained from Freedom Fuels, LLC (Mason City, Iowa) were added to the vessel, followed by the dropwise addition of 50 parts of 35% hydrogen peroxide at a rate sufficient to bring the reaction mixture to 90° C. Following completion of the reaction, the resulting glycerose liquor (Liquor A) was analyzed for reducing sugar content using the method of Example 1 and found to contain 16.8% glucose equivalents.

Using the method of Example 2, SBM was combined with sufficient Liquor A to add 2% glycerol or glycerol-derived oxidation products to the finished blend, and heated for 1, 5, 10 or 15 minutes. In comparison runs, SBM was combined with known solutions of glycerose dimer (Dimer A) or dihydroxyacetone dimer (Dimer B), using sufficient solution to add 0.5 wt. % dimer to the finished blend. These blends were heated using the method of Example 2 for 15 minutes. In a further comparison run, SBM was combined with 5 wt. % spent sulfite liquor (XYLIG™ lignosulfonate, LignoTech, USA, Rothschild, Wis.) and heated for 15 or 30 minutes. The resulting dried samples were sent to the FARME Institute for a 16 hour in situ protein degradation evaluation. The results are shown below in Table 3:

TABLE 3
Feed Treatments and Protein Evaluations

| | | | | Crude | |
|---|---|---|---|---|---|
| Run | | Dosage, % | Heating | Protein (CP), | RUP, % of |
| No. | Treatment | DM | Time, min | % of DM | CP |
| 3-1 | Liquor A | 2 | 1 | 48.6 | 67.9 |
| 3-2 | Liquor A | 2 | 5 | 50.6 | 66.7 |
| 3-3 | Liquor A | 2 | 10 | 49.3 | 71.9 |
| 3-4 | Liquor A | 2 | 15 | 49.7 | 75.4 |
| 3-5 | Dimer A | 0.5 | 15 | 50.4. | 47.5 |
| 3-6 | Dimer B | 0.5 | 15 | 50.2 | 41.0 |
| 3-7 | XYLIG | 5 | 15 | 50.5 | 61.9 |
| 3-8 | XYLIG | 5 | 30 | 47.1 | 72.3 |

The results in Table 3 show that addition of 2% Liquor A provided feed products whose RUP values were comparable to or better than the RUP values obtained using 5% spent sulfite liquor, and made using much shorter heating times. Treatment with glycerose dimer or with dihydroxyacetone dimer provided feeds having generally lower RUP values, suggesting that the test conditions did not permit the dimers to hydrolyze to their corresponding monomeric forms.

### EXAMPLE 4

### Catalyst and Chelant Evaluation

Varying amounts of ferrous sulfate heptahydrate catalyst and EDTA chelant were placed in a reaction vessel equipped with a thermometer, mechanical stirrer and cooling bath, and dissolved in 10 g deionized water. 100 grams of a biodiesel-derived glycerol waste stream containing 83% glycerol obtained from Minnesota Soybean Processors (Brewster, Minn.) were added to the vessel, followed by the addition at five minute intervals of five 1 mL aliquots of 35% hydrogen peroxide. The typical response to the initial 1 mL addition of hydrogen peroxide was an immediate color change from light yellow to reddish brown. At the same time an exothermic reaction occurred that increased the temperature by 6 to 9° C. This exothermic reaction was very rapid as the temperature began to decline after the first minute. During the second or third minute it was common to see small bubbles, presumed to be oxygen, forming in the glycerose liquor. The exothermic reaction appeared to take place before the appearance of bubbles. When the level of iron was 0.5 g or more the exotherm continued with each incremental addition of hydrogen peroxide. When iron was omitted, no heating occurred with addition of peroxide.

After 25 minutes, additional hydrogen peroxide was added to each reaction mixture in a continuous dropwise stream at approximately 1 nL/min while cooling the reaction vessel sufficiently to maintain the reaction mixture at 50 to 60° C., until a total of 44.4 mL (50 g) of hydrogen peroxide had been added. Effervescence was common to most treatments, with audible fizzing occurring as the bubbles broke the surface. This is most likely oxygen and represents a waste of hydrogen peroxide, the single most expensive ingredient. The peroxide might be used more efficiently if the addition rate were slower or if the reaction were carried out at elevated pressure. In a commercial setting, a slower addition rate might provide additional time for the removal of heat.

Acidity was measured using a pH meter. Following completion of the reaction, the resulting glycerose liquors (Liquors B through H) were analyzed for reducing sugar content using the method of Example 1 but with comparison to a glycerose standard rather than a glucose standard. The liquors were also analyzed for carboxyl content using conductometric titration.

Using the method of Example 2, a 10 minute heating time and an add-on rate sufficient to provide 1% glycerol-derived product in the finished blends, glycerose liquors B through G were used to treat SBM and determine their potential to generate RUP. A SBM sample was also treated with sufficient water to increase the moisture content to 20% and heated for ten minutes. The resulting samples were exposed to air to allow cooling and drying, and sent to the FARME Institute for a 16 hour in situ protein degradation evaluation. The results are shown below in Table 4:

TABLE 4
Feed Treatments and Protein Evaluations

| | | | | | | | | RUP, |
|---|---|---|---|---|---|---|---|---|
| Run | | Fe++ | | | | Glycerose | | % of |
| No. | Treatment | Catalyst, g | EDTA, g | pH | Exotherm | Equivalent, wt. % | Carboxyl, % | CP |
| 4-1 | Liquor B | 0.5 | — | 2.15 | Strong | 17.4 | 3.2 | 74.6 |
| 4-2 | Liquor C | 1.0 | — | 2.09 | Strong | 17.1 | 2.5 | 76.0 |
| 4-3 | Liquor D | 2.0 | — | 1.93 | Strong | 14.8 | 3.5 | 69.8 |
| 4-4 | Liquor E | 0.5 | 1.0 | 2.45 | Strong | 18.6 | 1.7 | 70.4 |
| 4-5 | Liquor F | 1.0 | 1.0 | 2.53 | Strong | 17.7 | 1.8 | 66.7 |
| 4-6 | Liquor G | 0.25-0.35 | — | 1.77 | Mild | 0.9 | 9.4 | 51.1 |
| 4-7 | Water | — | — | — | None | — | — | 42.8 |

Increasing the level of ferrous sulphate (Fe++ catalyst) tended to reduce the observed aldehyde level (reported in Table 4 as the Glycerose Equivalent) and lower the pH, suggesting that some of the aldehyde may have been further oxidized to a carboxylic acid. Addition of EDTA tended to increase the observed aldehyde level, raise the pH, and reduce the carboxyl level, all favourable responses. EDTA additions thus seemed to reverse the effect of high catalyst levels, suggesting that the EDTA was chelating the ferrous ion. A combination of 0.5 g ferrous sulfate and 1.0 g of EDTA appeared to provide a desirable level of RUP and suggested that the chelated iron remained effective as a catalyst. A reduced RUP value was observed for SBM treated with Liquor F (made using 1 g each of catalyst and EDTA). Increased glycerose equivalent and RUP levels were observed for SBM treated with Liquor E (made using 0.5 g catalyst and 1 g EDTA), suggesting additional improvements might be obtained by adjusting the catalyst and EDTA amounts or the ratio of EDTA to catalyst. For example, 100 g crude glycerol and 10 g water might be combined with about 0.5 g catalyst and 0.7 g EDTA to provide a starting mixture containing about 900 ppm iron and about 0.6 wt. % EDTA.

Biodiesel-derived glycerol often contains a small amount of fatty acid, and the Minnesota Soybean Processors glycerol used in this Example is said to include a 0. 15% fatty acid content. Fatty acids may be capable of combining with divalent cations to form insoluble salts analogous to those responsible for bath tub ring formation. In some of the runs noted above a reddish brown scum was observed to have adhered to the sides of the reaction vessel, possibly due to a combination of fatty acid(s) (e.g., linoleic acid) with the ferrous ion catalyst. If carried out on a commercial scale a similar combination might lead to formation of greasy globs capable of blocking filters or nozzles. The addition of EDTA appeared to reduce or eliminate scum formation and was thought to be due to chelation of the ferrous ion and consequent reduction or prevention of its interaction with fatty acids.

For Liquor G, the low initial catalyst level appeared to provide a milder exotherm, and the temperature began to decline 30 minutes after start of the initial peroxide addition even as the peroxide addition continued. This suggested that the additional peroxide was not reacting due to iron depletion or deficiency in the reaction mixture. At t=35 minutes, a further 0.1 g of ferrous sulfate was added to the reaction mixture. Over the next 5 minutes no further peroxide was added but the temperature increased, indicating that unreacted peroxide had been present in the liquor. The peroxide addition was resumed at t=40 minutes but the temperature began to decline again after t=50 minutes. The concentrations of ferrous ion when the temperature began to decline were 430 and 520 ppm, suggesting that for this reaction vessel and under these conditions, a possible minimum catalyst level might be about 500 to about 600 ppm. Analysis of Liquor G showed it to have a high carboxyl content and very little aldehyde, suggesting that a low ferrous ion concentration may favor further oxidation of aldehydes and formation of carboxylic acids.

### EXAMPLE 5

The method of Example 4 was repeated using the Freedom Fuels, LLC glycerol waste stream employed in Example 3. The resulting dried samples were sent to the FARME Institute for a 16 hour in situ protein degradation evaluation. The results are shown below in Table 5:

TABLE 5
Feed Treatments and Protein Evaluations

| Run | | Fe++ | | | Glycerose | | RUP, % of |
|---|---|---|---|---|---|---|---|
| No. | Treatment | Catalyst, g | EDTA, g | pH | Equivalent, wt. % | Carboxyl, % | CP |
| 5-1 | Liquor H | 0.5 | — | 2.32 | 18.3 | 2.5 | 78.4 |
| 5-2 | Liquor I | 1.0 | — | 2.00 | 16.9 | 2.3 | 77.9 |
| 5-3 | Liquor J | 2.0 | — | 1.88 | 16.7 | 2.5 | 76.1 |
| 5-4 | Liquor K | 0.5 | 1.0 | 2.70 | 19.2 | 1.7 | 71.5 |
| 5-5 | Liquor L | 1.0 | 1.0 | 2.47 | 16.9 | 2.2 | 57.9 |

Like the results in Table 4, the results in Table 5 demonstrated high RUP levels.

### EXAMPLE 6

### Fodder Heating Time Effects

Liquor E from Table 4 and Liquor K from Table 5 were combined with SBM using the method of Example 2 and heated for 1, 10 or 30 minutes. During the one minute heating time, the liquor-SBM mixture temperatures were observed to increase from 25 to about 95° C. A SBM sample was also treated with water to bring the total moisture level to 20% and heated for one minute. The resulting dried samples were sent to the FARME Institute for a 16 hour in situ protein degradation evaluation. The results are shown below in Table 6:

TABLE 6
Effect of Fodder Heating Times

| Run | | | RUP, % of |
|---|---|---|---|
| No. | Treatment | Heating Time, min | CP |
| 6-1 | Liquor E | 1 | 61.4 |
| 6-2 | Liquor E | 10 | 71.5 |
| 6-3 | Liquor E | 30 | 70.4 |
| 6-4 | Liquor K | 1 | 50.9 |
| 6-5 | Liquor K | 10 | — |
| 6-6 | Liquor K | 30 | 71.5 |
| 6-7 | Water | 1 | 42.8 |

As shown in Table 6, both liquors provided feed products with increased RUP versus a water only control, even after as little as one minute of heating. This demonstrates that that the disclosed glycerose liquors can provide RUP improvements with low heating times and temperatures.

### EXAMPLE 8

### Glycerol Processing Temperatures

Ferrous sulfate heptahydrate (0.5 g) was combined with 1.0 g tetra sodium EDTA and 10 mL of distilled water in a 500 mL round bottom flask, followed by the addition of 100 g crude glycerol from Minnesota Soybean Processers. The flask was placed in a water bath to warm the mixture and maintain various target processing temperatures. The flask was fitted with a thermometer and mechanical stirrer. Fifty grams of 35% hydrogen peroxide were added to the flask in a dropwise manner over a period of three hours. This process was repeated five times at target processing temperatures of 40, 60, 70, 80, and 88° C., yielding glycerose liquors containing about 50% glycerol products. The glycerose equivalents in each glycerose liquor were determined by titration against known standards and reported as a percentage of total liquid weight. Yields tended to decline with increasing processing temperature and the decline became severe when the processing temperature exceeded 80° C. At the highest processing temperature, strong effervescence was observed with each added drop of peroxide. Under these circumstances, the peroxide may be decomposing to form oxygen before it can react with glycerol. Based on this work and subsequent confirmatory runs, processing temperatures of about 50 to about 60° C. appear to provide especially desirable glycerose yields.

TABLE 8
Effect of Glycerol Processing Temperatures

| | Process | | Glycerose |
|---|---|---|---|
| Run No. | Temperature, ° C. | pH | Equivalent, wt. % |
| 8-1 | 40 | 2.8 | 16.7 |
| 8-2 | 60 | 2.7 | 14.3 |
| 8-3 | 70 | 2.8 | 14.2 |
| 8-4 | 80 | 2.8 | 13.5 |
| 8-5 | 88 | 2.7 | 8.6 |

### EXAMPLE 9

### Further Catalyst and Chelant Evaluation

Using the method of Example 4, varying amounts of ferrous sulfate heptahydrate catalyst and EDTA chelant were reacted with 100 grams of the Minnesota Soybean Processors biodiesel-derived glycerol waste stream. The resulting glycerose liquors were combined with SBM as in Example 4 and Example 2, using a 10 minute feed heating time. The results are shown below in Table 9:

TABLE 9
Feed Treatments and Protein Evaluations

| Run | | Fe++ | EDTA, | | Glycerose | RUP, % |
|---|---|---|---|---|---|---|
| No. | Treatment | Catalyst, g | g | pH | Equivalent, wt. % | of CP |
| 9-1 | Liquor M | 0.5 | 0.5 | 2.9 | 21.1 | 62.2 |
| 9-2 | Liquor N | 1.0 | 1.0 | 3.1 | 19.7 | 71.8 |
| 9-3 | Liquor O | 0.6 | 1.0 | 3.2 | 20.3 | 64.4 |
| 9-4 | Liquor P | 0.5 | 0.8 | 3.1 | 20.6 | 61.4 |
| 9-5 | Liquor Q | 1.0 | 0.5 | 2.8 | 19.4 | 72.8 |
| 9-6 | Liquor R | 0.5 | 1.0 | 3.2 | 21.5 | 61.8 |
| 9-7 | Liquor S | 0.5 | 0.7 | 3.1 | 20.9 | 57.3 |
| 9-8 | Water | — | — | — | — | 47.2 |

In Run Nos. 9-2 and 9-3, a small quantity of black floating scum appeared in the 5 glycerose liquor. This is believed to be due to formation of an iron-fatty acid salt, and may have lowered the glycerose equivalent yield. The scum was eliminated when the ratio of sodium EDTA to ferrous sulfate heptahydrate exceeded about 8:5.

The Run No. 9-7 glycerose liquor (Liquor S) was stored for five months to evaluate its shelf stability, then applied along with water to a 100 g SBM sample in amounts sufficient to provide 1% glycerol-derived product in the finished blends and bring the total moisture level to 20, and heated for 10 or 30 minutes. The resulting feed products were sent to the FARME Institute for in situ determination of crude protein and RUP. The results are shown below in Table 10:

TABLE 10
Stored Glycerose Liquor Evaluation

| | Treatment, g | | | Roasting | |
|---|---|---|---|---|---|
| | Glycerose | | Treatment | Time, | RUP, % of |
| Run No. | Liquor | Water | Solution pH | min. | CP |
| 9-9 | 2.94 | 30.6 | 2.83 | 10 | 71.4 |
| 9-10 | 2.94 | 30.6 | 2.87 | 30 | 74.1 |

The results in Table 10 show that the glycerose liquor solution continued to provide bypass protein protection after lengthy storage.

### EXAMPLE 10

### Oxidant:Glycerol Ratio

Two hundred grams of a biodiesel-derived glycerol waste stream containing 82% glycerol obtained from Minnesota Soybean Processors and 5.6 g of a 40% tetrasodium EDTA solution (DISSOLVINE™ E-39, Akzo Nobel) were added to a round bottom flask. The flask was first agitated by hand and then stirred continuously using a magnetic stirrer. Ferrous sulfate heptahydrate (1.6 g) was dissolved in 5.0 g of warm water and the resulting solution was added to the flask. Hydrogen peroxide (35%) was added to the reaction flask using a burette. Initially, 2 mL increments were added rapidly at five minute intervals. When the hydrogen peroxide addition is initiated the reaction liquor is dense and viscous and the hydrogen peroxide may not mix well. Local concentrations may heat up such that the reaction liquor may boil and the peroxide will disproportionate. As further hydrogen peroxide is added the reaction liquor warms and become diluted, reducing its viscosity. Care was taken to provide strong mixing at the point of hydrogen peroxide addition. A thermometer was also placed in the reaction liquor and the temperature recorded at one-minute intervals. After 10 mL of peroxide had been added, the addition was changed to a continuous mode at a rate of about 1 mL per minute. During the continuous addition period water was placed in a containment vessel surrounding the flask. Approximately one liter of cool tap water was used. This was changed every time another 50 mL of peroxide had been added. Heat generation during a similar reaction was estimated to be approximately 730 kcal/liter of 35% hydrogen peroxide added to the reaction flask.

Sub-samples (25 mL) were pipetted from the reaction liquor at intervals corresponding to peroxide:glycerol (viz., oxidant:glycerol) ratios of 0.4, 0.6, 0.8, 0.9, 1.0, 1.1, and 1.2, on a liquid weight basis. These corresponded to collections made when 71, 103, 131, 144, 156, 166, and 176 mL of peroxide had been added. The sub-samples were weighed and stored for later testing. The pH of each sub-sample was also measured by inserting a pH instrument probe directly into the reaction liquor.

Two portions of glyceraldehyde (0.0400 and 0.0520 g) were dissolved in 200 mL of warm deionized water to make glyceraldehyde standards with concentrations of 200 and 260 mg/L. Five milliliter aliquots of each standard and a 5 mL aliquot of deionized water were pipetted into 25×200 mm PYREX™ test tubes to provide two standard solutions and a deionized water blank. Five mL of Copper Reagent were added to each test tube. The three tubes were heated in boiling water for 40 minutes and cooled. Each tube was treated by addition of 2 mL 2.5% KI and 1.5 mL 2N H₂SO₄. About 10 mL of 0.005 N sodium thiosulfate solution was added to each tube, followed by 4 mL of 0.4% dissolved starch, which turned the solution dark blue. Additional sodium thiosulfate was added to titrate the samples until the solutions were clear and colorless. The net difference between the volumes of titrant used for the glyceraldehyde standard solutions and the deionized water blank was used to calculate the titrant strength.

The glycerose liquor sub-samples were next titrated to determine their glycerose content. About 0.7 g of each liquor was weighed into a 500-mL volumetric flask and diluted with deionized water to volume. A 5 mL aliquot of this solution was transferred to a PYREX test tube, combined with Copper Reagent, heated for 40 minutes, and titrated with sodium thiosulfate as described above. The volume of titrant was recorded and used to calculate the amount of glycerose in each sub-sample.

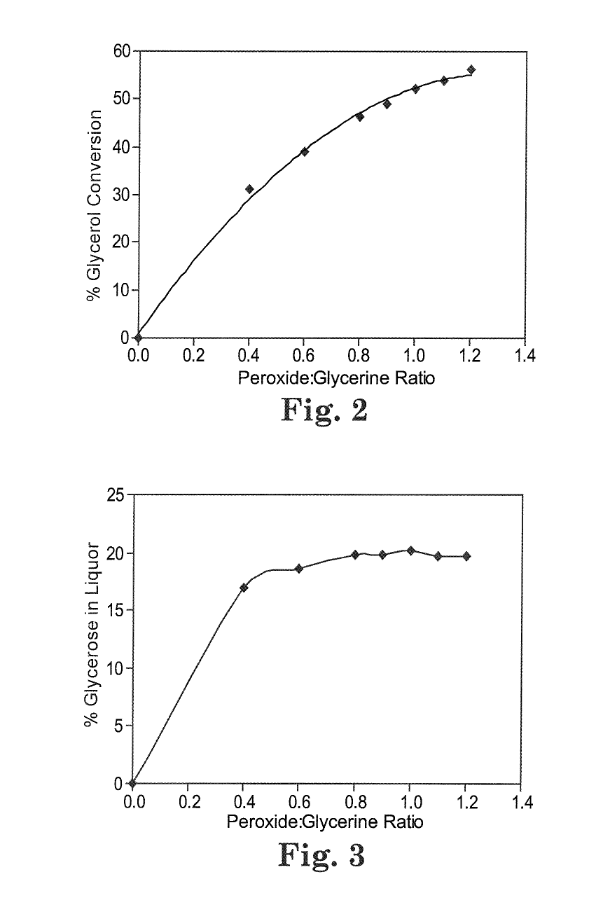

As shown in FIG. 1 and FIG. 2, the addition of hydrogen peroxide to glycerol generates increasing amounts of glycerose (FIG. 1) but also dilutes the reaction liquor (FIG. 2). The rate of glycerose production and liquor dilution may be balanced so that the percentage of glycerose in the reaction liquor reaches a plateau (FIG. 2). The shape and nature of the plateau may be altered by using a more concentrated (e.g., 50%) or less concentrated (e.g., 30%) oxidant solution. The oxidant:glycerol ratio may be altered over wide ranges as desired (e.g., 60 to 100 parts or 80 to 100 parts of hydrogen peroxide per 100 parts of glycerol) and may affect variables including production cost, glycerose yield, product viscosity and acidity. The occurrence of possible side reactions may also be taken into account. For example, if one mole of hydrogen peroxide reacted with one mole of glycerol to produce one mole of glycerose there would be 100% conversion of glycerol to glycerose at an oxidant:glycerol ratio of 0.865. However, for the reactions shown in FIG. 1 only about 50% conversion was observed at that ratio. Some portion of the hydrogen peroxide may have decomposed to oxygen without reacting with glycerol, and another portion of the hydrogen peroxide may have reacted with glycerose to oxidize it further. The production of additional glycerose at oxidant:glycerol ratios >0.9 indicates the continued presence of unreacted glycerol.

The sub-samples were also used to treat SBM to determine their potential to generate RUP. In each case 150 g of SBM was combined, using the method of Example 2, with an amount of sub-sample sufficient to provide 1% glycerol-containing product in the finished blend. The samples contained 80% DM and were heated in a 105° C. oven for 9 minutes. The resulting feed products were sent to the FARME Institute for in situ determination of crude protein and RUP. The results are shown below in Table 11:

TABLE 11
Oxidant:Glycerol Ratio Protein Evaluations

| Run | | RUP, % of |
|---|---|---|
| No. | Oxidant:Glycerol Ratio | CP |
| 11-1 | 0.40 | 61.5 |
| 11-2 | 0.60 | 63.1 |
| 11-3 | 0.80 | 70.7 |
| 11-4 | 0.90 | 68.2 |
| 11-5 | 1.00 | 67.5 |
| 11-6 | 1.10 | 75.0 |
| 11-7 | 1.20 | 67.8 |

Having thus described the preferred embodiments of the present invention, those of skill in the art will readily appreciate that the teachings found herein may be applied to yet other embodiments within the scope of the claims hereto attached. The complete disclosures of all patents, patent documents, and publications are incorporated herein by reference as if individually incorporated.

We claim:

1. A method for making glycerose, which method comprises the steps of:
a. obtaining glycerol from a biodiesel manufacturing process; and
b. partially oxidizing the glycerol to provide a glycerose liquor containing glycerose and other side products of the oxidation reaction.

2. A method according to claim 1 wherein the glycerol comprises crude glycerin.

3. A method according to claim 1 wherein the glycerol is partially oxidized using an oxidizing agent in the presence of a suitable catalyst.

4. A method according to claim 1 wherein the glycerol is partially oxidized using hydrogen peroxide and ferrous sulfate.

5. A method according to claim 1 wherein the glycerol is partially oxidized using about 500 to about 3000 ppm of an iron-containing catalyst.

6. A method according to claim 1 wherein the glycerol is partially oxidized using oxygen or air with water as a solvent.

7. A method according to claim 1 wherein the glycerol is partially oxidized using enzyme oxidation, bacterial fermentation, catalytic oxidation or combination thereof.

8. A method according to claim 1 wherein the glycerol is partially oxidized in the presence of a chelating agent.

9. A method according to claim 1 wherein the glycerol is partially oxidized in the presence of ethylene diamine tetraacetic acid.

10. A method according to claim 1 comprising partially oxidizing a solution containing more than 10 wt. % and less than 85 wt. % glycerol.

11. A method according to claim 1 comprising partially oxidizing crude glycerin containing about 45 to about 85 wt. % glycerol.

12. A method according to claim 1 comprising partially oxidizing the glycerol at less than 90° C.

13. A method according to claim 1 comprising partially oxidizing the glycerol at about 50 to about 60° C.

14. A method according to claim 1 comprising partially oxidizing the glycerol under acidic conditions.

15. A method for making glycerose, which method comprises the step of combining a glycerol solution with an oxidizing agent and a ferrous ion catalyst to provide a reaction mixture containing more than 10 wt. % glycerol and less than 1000 ppm catalyst.

16. A method according to claim 15 wherein the glycerol solution comprises crude glycerin from biodiesel manufacturing.

17. A method according to claim 15 wherein the glycerol solution contains about 45 to about 85 wt. % glycerol.

18. A method according to claim 15 comprising partially oxidizing the glycerol using hydrogen peroxide and ferrous sulfate.

19. A method according to claim 15 comprising partially oxidizing the glycerol using at least about 500 ppm of the ferrous ion catalyst.

20. A method according to claim 15 comprising partially oxidizing the glycerol at less than 90° C.

21. A method according to claim 15 comprising partially oxidizing the glycerol at about 50 to about 60° C.

22. A method according to claim 15 comprising partially oxidizing the glycerol under acidic conditions.

23. A method for making glycerose, which method comprises the step of combining a glycerol solution with an oxidizing agent, a catalyst and a chelating agent.

24. A method according to claim 23 wherein the wherein the glycerol solution comprises crude glycerin from biodiesel manufacturing.

25. A method according to claim 23 wherein the glycerol solution contains about 45 to about 85 wt. % glycerol.

26. A method according to claim 23 comprising partially oxidizing the glycerol using hydrogen peroxide and ferrous sulfate.

27. A method according to claim 23 comprising partially oxidizing the glycerol using about 500 to about 3000 ppm of the catalyst.

28. A method according to claim 23 comprising partially oxidizing the glycerol at less than 90° C.

29. A method according to claim 23 comprising partially oxidizing the glycerol at about 50 to about 60° C.

30. A method according to claim 23 wherein the chelating agent comprises ethylene diamine tetraacetic acid.

31. A method according to claim 23 comprising using sufficient chelating agent to reduce scum formation.

32. A method according to claim 23 comprising using sufficient chelating agent to eliminate scum formation.

33. A method according to claim 23 wherein the glycerol solution further comprises fatty acid(s).
